(19) United States
(12) Patent Application Publication
(10) Pub. No.: US 2007/0129644 A1
(43) Pub. Date: Jun. 7, 2007

(54) Sleep disorder screening program

(76) Inventors: Glenn Richards, Kenthurst (AU); Rhonda Russo, Rydalmere (AU)

Correspondence Address: GOTTLIEB RACKMAN & REISMAN PC, 270 MADISON AVENUE, 8TH FLOOR, NEW YORK, NY, 100160601, US

(21) Appl. No.: 11/292,683

(22) Filed: Dec. 2, 2005

(51) Int. Cl. A61B 5/08

(52) U.S. Cl. 600/529; 600/323

(57) ABSTRACT

A method of screening for the risk of a sleep disorder such as obstructive sleep apnea proceeds by first providing a sleep health education to a member of an organization, offering and following up by providing a portable screening device for use by the member at home, which monitors a respiratory parameter such as airflow and/or oxygen saturation. The data from the monitoring is then analysed to determine the presence of the sleep disorder and the results are then reported.

### I. FIELD OF THE INVENTION

This invention relates to a screening program for risk of a sleep disorder, preferably for risk of Obstructive Sleep Apnea (OSA).

### II. BACKGROUND OF THE INVENTION

A. The Problem addressed by the Invention

### i. Description of the OSA Syndrome

Obstructive Sleep Apnea (OSA) Syndrome is a sleep and breathing disorder where the upper airway partially or completely occludes during sleep. The presence of OSA Syndrome is defined as at least five obstructed breathing episodes per hour of sleep together with daytime sleepiness symptoms. Some studies estimate that OSA Syndrome affects approximately 2-4% of the general population. There are a number of factors that increase the risk of OSA Syndrome including age, sex and weight. The prevalence in males and females over 30 years of age may be closer to 24% and 9% respectively. Thus this disorder has a significant impact on society. However, the diagnosis and treatment rates for this disorder are currently very low.

Excess tissue in the upper airway and physical abnormalities worsen OSA. During sleep, especially in REM sleep, bodies relax, the muscle tissues like the tongue and soft palate lose their slight rigidity, and the airway collapses. When these tissues obstruct the upper airway completely, they prevent breathing and can actually begin to suffocate the sleeper. The sleeper wakes up enough to regain control of the upper airway, breathe again, and then fall back to sleep. This happens from dozens to hundreds of times per night for people with OSA, although they usually don't remember waking up.

Each obstruction deprives the body of oxygen and does not allow it to rid itself of carbon dioxide that it would normally exhale. When the body sets off “alarms” that it 3869/270 needs more oxygen, the brain wakes the sleeper, breathing resumes, and the individual falls back to sleep until the next obstruction occurs. These obstructions increase heart rate, raise blood pressure, and eventually blunt the body's automatic response system, allowing increasingly more severe apneas and hypopneas.

OSA may have a significant impact on an individual's health. Due to the disruption in sleep a common symptom of OSA is daytime sleepiness. Daytime sleepiness may impact an individual's performance and/or ability to control machinery. For example daytime sleepiness caused by OSA may impair an individual's ability to drive a car and may be a factor in car accidents.

### ii. Current Treatment Methods

The current method of treatment for OSA is positive airway pressure delivered to an individual's airways while they are asleep. Colin Sullivan invented the use of nasal Continuous Positive Airway Pressure (CPAP) to treat Obstructive Sleep Apnea (OSA) see U.S. Pat. No. 4,944,310. The treatment generally provides a continuous supply of air or breathable gas from a blower or flow generator to a patient via an air delivery conduit and a patient interface, such as a full-face or nasal mask or nasal prongs. The air or breathable gas is commonly delivered at a pressure of 4 cmH₂0 to 20 cmH₂0 and acts as a splint to hold the airway open during sleep.

Further developments of CPAP have provided automatically adjusting devices for use in a patient's home. The automatically adjusting device will raise and/or lower the treatment pressure based on indications of OSA, such as snoring. Such CPAP devices are sometime generically referred to as Automatic Positive Airway Pressure (APAP) devices. An example of an APAP device is the ResMed AutoSet® Spirit™. See U.S. Pat. Nos. 5,245,995; 6,279,569; 6,398,739; 6,425,395; 6,502,572; 6,532,959 and 6,635,021.

Another type of nasal CPAP device provides a first pressure during inhalation (typically termed IPAP for Inspiratory Positive Airway Pressure) and a second, lower pressure during exhalation (typically termed EPAP for Expiratory PAP). Examples of these devices include the ResMed VPAP™ series (see U.S. Pat. Nos. 4,944,310, 6,213,119, 6,240,921, 6,705,315), and the Respironics BiPAP series. Bilevel CPAP devices may be prescribed for patients who do not comply with single pressure CPAP devices. Some patients perceive that the lower pressure during exhalation is more comfortable, at least while they are awake.

### iii. Current Diagnosis and Treatment Processes

The current diagnosis and treatment process for OSA generally begins when an individual or their bed partner complains of daytime sleepiness or excess snoring. In this scenario the individual would visit a GP for a referral to a sleep specialist. The individual would then make an appointment to visit the sleep specialist who would review the patient's symptoms and refer them to a sleep laboratory for diagnosis by polygraphic study (PG) or polysomnography (PSG). For diagnosis and a treatment plan to be established an individual needs to spend two nights in the sleep laboratory: the first night for diagnosis and the second night for therapy titration. Therapy titration identifies the correct treatment pressure required by the individual to overcome OSA, such that apneas are eliminated. The treatment pressure is usually in the range of 4-20 cmH₂0. Following diagnosis and therapy titration an individual may obtain an appropriate therapy device that is set to the identified treatment pressure. Unfortunately this process is time consuming and expensive. In view of this there are often extensive waiting periods, from 4 weeks to two years or more, for a PSG at a sleep laboratory. Furthermore, the system relies on the individuals recognising the symptoms and seeking medical advice. Currently the awareness level of this disorder is low which impacts the recognition of symptoms, diagnosis and ultimately treatment.

There are a number of community health screening programs currently available for various maladies. These programs are commonly offered to the general public at public locations such as pharmacies or may be offered at particular work sites. Blood cholesterol and blood glucose tests are routinely performed to assess an individual's risk of developing heart and blood vessel disease or diabetes respectively. The test is generally performed using a finger stick method, which involves taking a small blood sample from the individuals' fingertip and blotting the blood onto a disposable assessment strip. An analysis machine determines the individuals' blood cholesterol and/or blood glucose level. The test commonly takes about ten minutes to perform and provides a good indication of an individual's risk of developing heart disease or diabetes.

In contrast, the risk of having a sleep disorder requires an assessment to be performed whilst an individual is sleeping. Therefore quick screening programs at public locations are not feasible for sleep disorders.

It would be advantageous to provide an alternative method for screening individuals for OSA that was quicker and more economical.

### III. BRIEF DESCRIPTION OF THE INVENTION

An object of the present invention is to provide a low cost screening program for sleep disordered breathing.

Another object of the invention is to provide an employee benefit program that offers screening for sleep disorders.

A further object of the invention is to provide a screening program that has a higher success rate of finding people with sleep disorders than conventional programs.

Another object of the invention is to provide a screening program for risk of a sleep disorder in a comfortable sleeping environment, most preferably the sleeping environment would be in the individuals' own home.

Another object of the invention provides a method of increasing awareness of OSA by delivering an education and/or screening program to individuals through a work benefit scheme.

Another object of the invention is to provide a screening program to an organization, wherein the program includes:
1. providing sleep health education to the organization;
2. issuing a portable screening device for an individual of the organization to use at home, wherein the portable screening device is adapted to monitor at least one respiratory parameter, such as airflow rate, airflow volume, or oxygen saturation;
3. analysing the at least one respiratory parameter to determine the presence or absence of the sleep disorder; and
4. reporting the results to the individual.

In a preferred embodiment the sleep disorder is obstructive sleep apnea (OSA).

Preferably the education for individuals relating to sleep health provides information related to OSA and the benefits of treatment. In a more preferred embodiment the education program would include an initial assessment of the risk of having a sleep disorder for each individual of the organization. The initial assessment may include assessment questionnaires relating to an individuals sleepiness and/or health.

In one embodiment the education and/or initial assessment may be provided via computer using an internet or intranet link.

In another embodiment the education and/or screening program would be implemented as part of the benefits offered by an existing third party Health Screening Provider or by a new third party Health Screening Provider. The third party Health Screening Provider would have an alliance with the Sleep Disorder Company.

In another preferred embodiment a screening program would initially be offered to a selection of the members from an organization to allow initial evaluation of the program. For example the program may be offered to company executives or other management staff. Upon satisfactory completion of this initial screening program a wider screening program may be implemented.

The portable screening device is preferably small, portable and easy to use for the inexperienced user. The portable screening device monitors at least one respiratory parameter. In one preferred embodiment the portable device is a single channel parameter device such as the ResMed ApneaLink. However, other screening devices such as the MedCare Embletta™ may be used.

A further embodiment is a business method that would include additional awareness and screening programs to be offered to relatives and/or friends of the individual from the primary organization. Also an individual from the primary organization may elect to transfer its benefit to a family member rather than pursue the offer for itself.

Most preferably the business method would include the additional step of placing individuals identified as having the sleep disorder on a therapy trial. Most preferably the therapy trail would include the use of an auto-titrating pressure device, such as the ResMed AutoSet Spirit™. Using the data management capabilities of such a device will give an objective picture of the patient's condition.

Following the therapy trial the identified individual would be offered the opportunity to purchase a therapy device with added incentives for purchasing the therapy equipment, such as a therapy device, from the Sleep Disorder Company. For example, individual participants in the program may be issued with a loyalty card that provides discounts or bonuses in purchasing the required therapy equipment. Additional benefits such as an extended warranty and ongoing support, e.g. telephone support, may also be provided.

Preferably a copy of an individual's result will be sent to their General Practitioner or Primary Care Physician.

### IV. BRIEF DESCRIPTION OF FIGURES

FIG. 1 shows a flowchart outlining a screening method according to a first preferred embodiment of the invention.

FIG. 2 shows a flowchart outlining a screening method according to a second preferred embodiment of the invention.

FIG. 3a shows an outline of an embodiment the sleep heath benefit program procedures according to the invention.

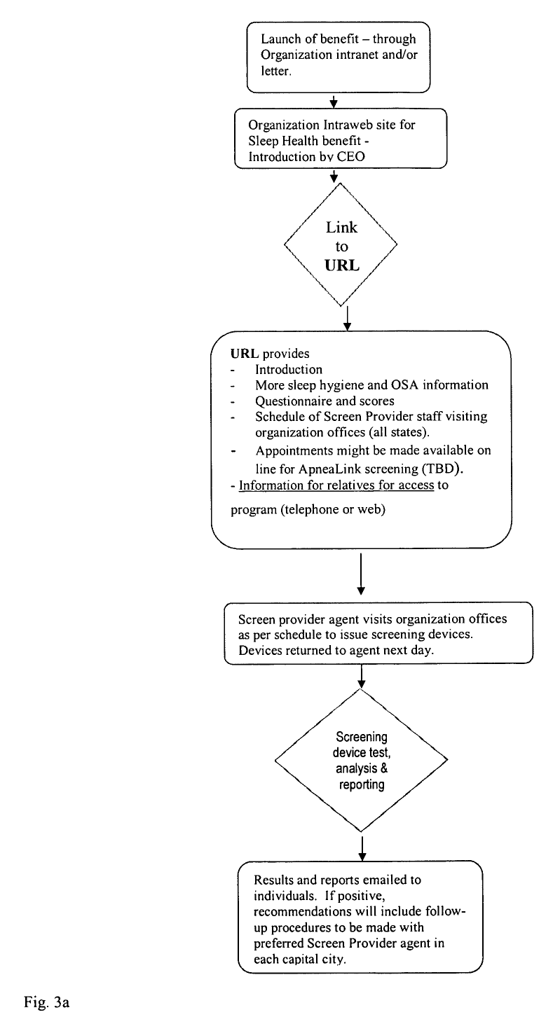

FIG. 3b shows an outline of another embodiment of the sleep health benefit procedures according to the invention.

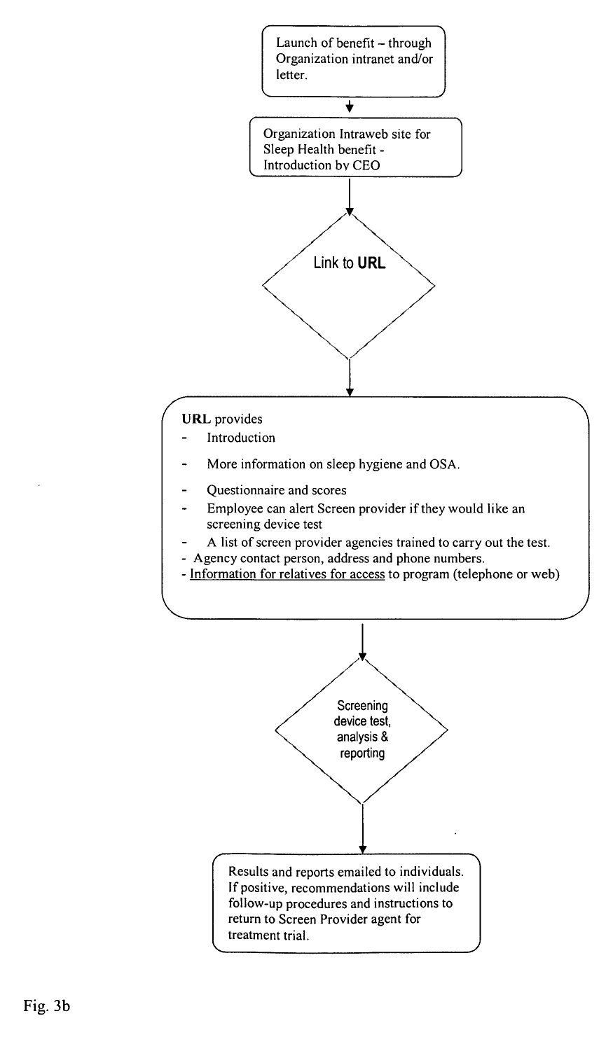

### V. DETAILED DESCRIPTION OF PREFERRED EMBODIMENTS OF THE INVENTION

A. Portable Screening Device

In accordance with an embodiment of the invention, people are loaned a screening device to take home so that they can be screened for a sleep disorder in their own home. In theory more representative results may be obtained when a person sleeps in their own home. Preferably the screening device is small, portable and easy to use. While full polysomnography provides a more detailed picture of the person's sleep, it requires trained staff to conduct such tests. Hence a device suitable for use with the invention must be simple enough for a member of the general public to use with basic training, and yet be sophisticated enough to provide a meaningful screening result. Because a system in accordance with the invention does not require trained medical staff to personally conduct all aspects of every screening test, the cost of screening is reduced. Suitable portable screening devices include ResMed's APNEA LIK and the MedCare EMBLETTA.

The ResMed ApneaLink® is a portable, mobile phone size device used for estimating the likelihood of a person having Sleep Disordered Breathing. It records high resolution flow data. Its storage capacity is sufficient for an overnight study.

The Embletta is a pocket-sized digital recorder for diagnosis of sleep disordered breathing (SDB). Embletta downloads a night's study using USB technology. The Embletta has an internal flash memory that can store comprehensive respiratory data. Embletta's diagnostic signals include position and activity, leg movement, oxygen saturation, pulse, oral flow, and respiratory effort signals through Respiratory Inductive Plethysmograph (RIP) sensors. The Embletta can be directly connected to a ResMed AutoSet® flow generator where flow, pressure, leak, and events from the AutoSet are added to the signals recorded by the Embletta. Exact nasal flow and pressure snore signals are obtained via a nasal cannula.

The portable screening device allows monitoring of at least one respiratory parameter. In one embodiment the portable screening device comprises a pressure transducer that is used to infer an individuals' airflow. In another embodiment the portable screening device further includes a position monitor. In a further embodiment the portable screening device includes an oximeter that allows measurement of oxygen saturation and allows pulse rate assessment. It is understood that the portable screening device may record other known respiratory parameters or combinations thereof.

Key/desirable characteristics of the screening device are the simplicity and accuracy of the device.

B. Education Program

An aspect of the invention is to provide an education program to people who may accept the offer of being screened. The education program teaches them about recognizing the symptoms and possible treatment using a CPAP device. The education program also informs people about how screening can be done at home. The education program can be conducted by a number of techniques including seminars, by email, and web pages. Email and web approaches are particularly advantageous because they are low cost.

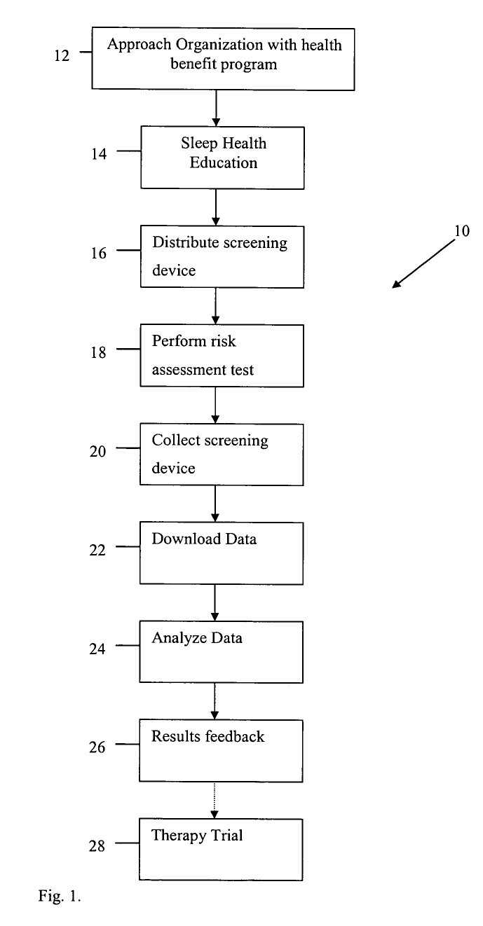

A preferred embodiment of the invention is illustrated in FIG. 1. In particular, step 14 shows providing sleep health education to increase awareness of sleep disorders, such as OSA, to the organization and its members. Step 14 may occur prior to, during or after acceptance of the program by the organization and more preferably is an on-going process step. For example some initial education may be provided to a portion of the organization staff to assist in understanding the program and the decision to pursue the program. Further education would then be provided to other members of the organization following acceptance of the program. During the sleep health education step 14 initial assessments relating to an individuals chance of having a sleep disorder may be conducted. Such initial assessments are preferably in the form of questionnaires related to sleepiness. Alternatively personal interviews may be conducted. The sleep health education step 14 promotes interest in the sleep disorder and assists in encouraging participation in the screening program.

C. Distribution & Collection of Screening Device

Should a person choose to take up the opportunity to be screened, then a low cost method is used to distribute and collect the screening device.

Individual barcodes or identifiers may be issued to individuals that participate in the screening program to assist in maintaining the privacy of an individual. The barcode or identifier may be issued together with or as part of a loyalty card that provides the individual with discounts and/or bonuses for purchasing therapy equipment if required following the screening program and therapy trial. The specific barcode or identifier may be used throughout the screening program to identify the particular individual. The specific barcode or identifier may also identify the source of the individual, for example the particular organization from which they originated.

In accordance with an embodiment of the invention as illustrated in FIG. 1., interested individuals may request a screen using the portable screening device; subsequently the individual collects the portable screening device 16 and preferably receives instruction on the use of the device. The risk assessment test 18 is performed overnight using the portable screening device. Most preferably the individual takes the portable screening device home to perform the risk assessment test, wherein the individual can self-fit the portable screening device and initiate the recording. Following the risk assessment test the device is returned 20 to the screen provider to download the data 22 and subsequently data analysis 24. The screen provider may be a representative from the Sleep Disorder Company or third party Health Screen Providers with alliances to the Sleep Disorder Company.

D. Analysis and Reporting of Results

Data must be transferred from the screening device for analysis. In one form, data is downloaded from the device via a USB port. In other forms of the invention, the Internet is used to transfer data back to a data center, for example, via a modem. Alternatively the information may be stored on a secure Internet site that is accessible by authorized personnel only. In some forms of the invention, data is continuously transmitted from the screening device and not stored or only backed up in the screening device. The data transfer is preferably performed under secure conditions to maintain the privacy of individuals' results.

In a preferred form of the invention, as shown in FIG. 1, the data is downloaded to the Sleep Disorder Company where, preferably trained clinicians perform the data analysis 24 to ensure dependability and consistency in the results. A report is generated based on the analyzed results. The results are feedback 26 to the individual generally in the form of a report although other formats may be used, such as personal consultation. The report is distributed to the individual and optionally the individual's selected General Practitioner or Primary Care Physician. The distribution process may include any known distribution process for example email, general mail or during a consultation.

Following the identification of a positive result for the tested sleep disorder an individual may decide to pursue a therapy trial 28. The therapy trial will preferably include the use of an auto-titrating pressure device, such as the ResMed AutoSet Spirit™. (See U.S. patents cited supra.) Generally an individual will be required to visit their General Practitioner or Primary Care Physician prior to commencing a therapy trial 28.

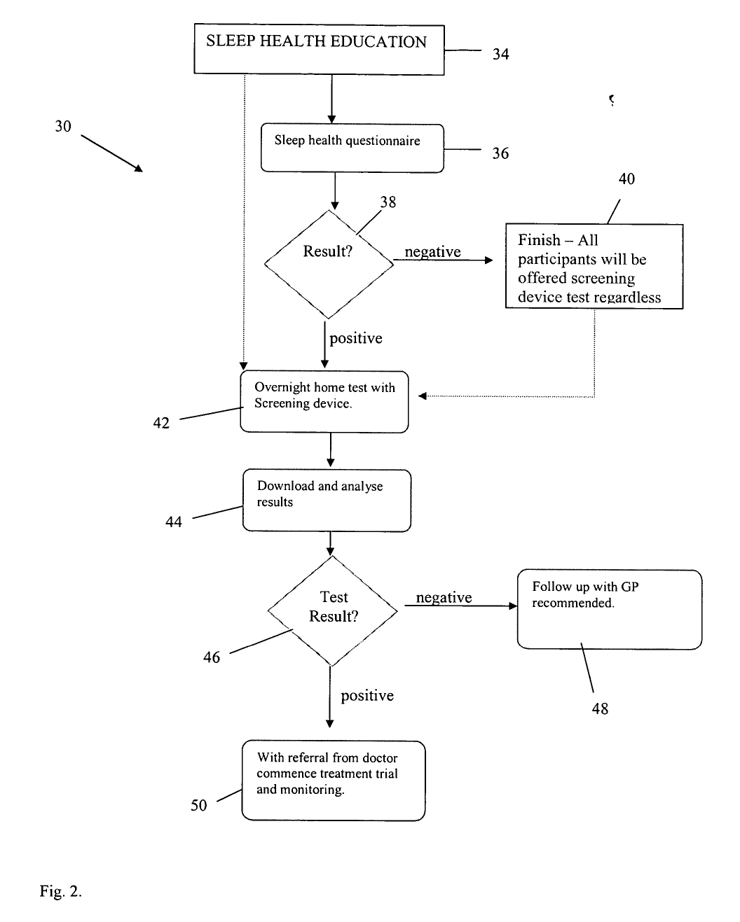

FIG. 2 shows a flow chart outlining the steps in a sleep disorder screening program according to a preferred embodiment of the invention. As described above the program may commence with sleep health education 34 and may lead to sleep health questionnaires 36 to provide an initial assessment of an individual's chance of having a sleep disorder. The individual may decide to pursue an overnight home test with a portable screening device 42 whether their questionnaire results are positive or negative. However, if the results are negative the individual may elect to finish the program at this stage 40. Alternatively the individual may elect not to participate in the sleep health questionnaire and immediately select screening with an overnight portable screening device 42.

Following the overnight risk assessment test 42 the results are downloaded and analyzed 44 as discussed previously. The reported results and recommendations to the individual will be determined from the outcome of the screening results. For a negative sleep disorder result the individual may be referred to their General Practitioner or Primary Care Physician if any symptoms persist. Alternatively if the results are positive the individual will be encouraged to obtain a referral from their General Practitioner prior to commencement of a therapy trial and monitoring. The service provider will provide information and assistance in implementing the therapy trial. Thus encouraging good customer relations with the individual. In some countries such as the United States individuals' require a prescription or referral from their Primary Care Physician before therapy may commence. In other countries, such as Australia, a referral from a General Practitioner is not essential for commencement of therapy. However, for reimbursement costs from a Private health insurance company a General Practitioner's referral is required.

E. Health Benefit Program

FIG. 1 shows a flowchart outlining a screening method 10 according to a first preferred embodiment of the invention. The initial step 12 is to approach a target organization, such as a corporate company, to offer a sleep health benefit program.

Advantageously this system provides a quick and efficient diagnosis system that offers increased awareness of sleep disorders and faster initiation of therapy times if required. The enhanced awareness promotes the Sleep Disorder Companies business profile and encourages good customer relations leading to higher customer potential and ultimately increased business sales.

The data obtained may also be gathered to provide statistics relating to the information initially provided by individuals, such as the results of initial screening questionnaires, and the actual determined risk of OSA. This data may be used to improve the screening program and consequently the chances of identifying individuals at risk of having OSA.

Additionally as the present health screening benefit program provides inclusion of an individual's General Practitioner or Primary Care Physician the awareness of sleep disorders is raised with these important health professionals. The information sent to an individual's General Practitioner or Primary Care Physician would be informative about sleep disorders and the health risks involved. Ultimately this may led to further patient/customer referrals to the Sleep Disorder Companies products and services by the General Practitioner. Information relating to OSA may be sent to General Practitioners or Primary Care Physicians independently from the individuals screening results. Screened individuals are encouraged to provide information relating to their General Practitioner or Primary Care Physician. This information may be compiled in a database system together with the details relating to what and when information relating to OSA was sent to the General Practitioner or Primary Care Physician. This would prevent repeatedly supplying the same information to the same General Practitioner or Primary Care Physician and may increase the awareness of OSA in a broader range of health professionals.

While the invention has been described in connection with what are presently considered to be the most practical and preferred embodiments, it is to be understood that the invention is not to be limited to the disclosed embodiments, but on the contrary, is intended to cover various modifications and equivalent arrangements included within the spirit and scope of the invention.

What is claimed is:

1. A method of screening for risk of a sleep disorder comprising
a) providing sleep health education to at least one member of an organization;
b) offering a screening program to the at least one member;
c) upon acceptance of the offer issuing a portable screening device to the at least one member, wherein the portable screening device is adapted to monitor at least one respiratory parameter;
d) analysing the at least one respiratory parameter to screen for the risk of the sleep disorder; and
e) reporting the results.

2. The method according to claim 1 wherein the method is performed as part of an employee health benefits program.

3. The method of low cost screening according to claim 1 wherein the sleep disorder is obstructive sleep apnea.

4. The method of low cost screening according to claim 1 or 2 wherein the method further includes providing an initial assessment for the risk of the sleep disorder for the at least one member.

5. The method of low cost screening according to claim 3 wherein the initial assessment includes the at least one member completing a questionnaire related to sleepiness and/or health.

6. The method of low cost screening according to claim 3 wherein the initial assessment is a self-assessed questionnaire.

7. An employee health benefit program comprising
a) providing sleep health education to at least one member of an organization;
b) offering a screening program to the at least one member;
c) upon acceptance of the offer issuing a portable screening device to the at least one member, wherein the portable screening device is adapted to monitor at least one respiratory parameter;
d) analysing the at least one respiratory parameter to screen for the risk of the sleep disorder; and
e) reporting the results to the at least one member.

8. A method of selecting participants for a sleep disorder screening program comprising
a) providing sleep health education program to at least one member of an organization, the education program including presenting identification characteristics of the sleep disorder;
b) based on the education program the at least one member selects at least one person to be offered a sleep disorder screen;
c) offering the sleep disorder screen to the at least one selected person;

9. The method according to claim 8 further comprising the following steps:
d) upon acceptance of the offer issuing a portable screening device to the at least one member, wherein the portable screening device is adapted to monitor at least one respiratory parameter;
e) analysing the at least one respiratory parameter to screen for the risk of the sleep disorder; and
f) reporting the results to the at least one selected person.

10. A screening program for risk of a sleep disorder, wherein the program includes:
providing sleep health education;
issuing a portable screening device for an individual to use at home, wherein the portable screening device is adapted to monitor at least one respiratory parameter, selected from the group consisting of airflow rate, airflow volume, or oxygen saturation;
analyzing the at least one respiratory parameter to determine the presence or absence of the sleep disorder; and
reporting the results to the individual.

11. The screening program of claim 1 wherein the sleep disorder is obstructive sleep apnea (OSA).

12. The screening program of claim 10 wherein the sleep health education for individuals provides information related to OSA and the benefits of treatment.

13. The screening program of claim 12, wherein the education program includes an initial assessment of the risk of having a sleep disorder.

14. The screening program of claim 13, wherein the education and/or initial assessment is provided via computer using an internet or intranet link.

15. The screening program of claim 13, wherein the screening program is part of benefits offered by an existing third party Health Screening Provider.

16. The screening program of claim 10, wherein the portable screening device is a single channel parameter device.

17. The screening program of claim 10 wherein the additional step of placing individuals identified as having the sleep disorder on a therapy trial.

18. The screening program of claim 17 wherein, the therapy trail includes the use of an auto-titrating pressure device.

19. A screening program for assessing a risk of a sleep disorder wherein
people are loaned a screening device to take home
recording at least one respiratory parameter,
download a night's study for analysis,
wherein the recorded respiratory parameters are selected from the group consisting of nasal flow and pressure snore signals, breathing sounds, respiratory flow, position, activity, leg movement, oxygen saturation, pulse, oral flow, and respiratory effort signals.

20. The screening program of claim 19 wherein nasal flow is obtained via a nasal cannula.

21. The screening program of claim 19 wherein the screening device comprises a pressure transducer that is used to infer an individuals' airflow.

22. The screening program of claim 21 wherein the portable screening device further includes a position monitor.

23. The screening program of claim 21 wherein the portable screening device includes an oximeter that allows measurement of oxygen saturation and allows pulse rate assessment.

24. The screening program of claim 19 employing barcodes or identifiers for individuals that participate in the screening program to assist in maintaining the privacy of an individual.

25. A screening program for risk of a sleep disorder comprising
providing a portable screening device for use at home by an individual
performing a risk assessment test overnight using the portable screening device,
returning the device to the provider
downloading data from the device
analyzing the data.

26. The screening program of claim 25 wherein the step of downloading the data is via a USB port or by via the internet back to a data center.

27. The screening program of claim 25 wherein the data is stored on an Internet site.

28. The screening program of claim 25 wherein the data is continuously transmitted from the screening device and not stored in the screening device.

29. The screening program of claim 25 generating a report based on analyzed results,
transmitting the report to the individual's selected General Practitioner or Primary Care Physician.

30. The screening program of claim 25 wherein, a positive result is followed by a therapy trial comprising the use of an auto-titrating pressure device.

31. A sleep disorder screening program comprising
a sleep health education,
an overnight home test with a portable screening device
downloading the results of the home test,
analyzing the results of the home test,
commencement of a therapy trial and monitoring.
